(19) United States
(12) Patent Application Publication
(10) Pub. No.: US 2024/0033414 A1
(43) Pub. Date: Feb. 1, 2024

(54) WEARABLE NEGATIVE-PRESSURE THERAPY SYSTEMS AND APPARATUSES AND RELATED METHODS

(71) Applicant: KCI Manufacturing Unlimited Company, Westmeath (IE)

(72) Inventors: Robert HOWARD, San Antonio, TX (US); Christopher Brian LOCKE, San Antonio, TX (US)

(21) Appl. No.: 18/265,568

(22) PCT Filed: Nov. 16, 2021
(86) PCT No.: PCT/IB2021/060598

§ 371 Date: Jun. 6, 2023

Related U.S. Application Data
(60) Provisional application No. 63/123,376, filed on Dec. 9, 2020.

(51) Int. Cl. A61M 1/00

(52) U.S. Cl. CPC A61M 1/912; A61M 1/962; A61M 2205/8206; A61M 2205/02

(57) ABSTRACT

An apparatus for treating a tissue site with negative pressure, the apparatus having a dressing, a dressing interface fluidly coupled to the dressing, and a conduit comprising a first end and a second end. The first end may be fluidly coupled to the dressing interface. The apparatus may also comprise a conduit chamber configured to retain at least a portion of the conduit and to allow the second end of the conduit to be extended from the dressing interface.

### CROSS-REFERENCE TO RELATED APPLICATIONS

This application is a U.S. National Stage entry of International Patent Application No. PCT/IB2021/060,598, filed on Nov. 16, 2021, which claims the benefit of priority to U.S. Provisional Application No. 63/123,376, filed on Dec. 9, 2020, which is incorporated herein by reference in its entirety.

### TECHNICAL FIELD

The invention set forth in the appended claims relates generally to tissue treatment systems and more particularly, but without limitation, to wearable negative-pressure therapy systems and apparatuses, and methods related to the same.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful wearable systems and apparatuses, and methods related to the same for the provision of negative-pressure therapy are set forth in the appended claims. Illustrative embodiments are also provided to enable a person skilled in the art to make and use the claimed subject matter.

For example, in some embodiments, the wearable systems and apparatuses, and methods related may include a single “peel-and-place” dressing that is customizable based upon the needs of a particular therapeutic environment, for example, based upon the location of a tissue site that will receive the therapy. Additionally, the dressing may provide an integrated negative-pressure conduit capable of being extended to various lengths. The dressing may also allow a negative pressure device or unit to be affixed to the user via a “skin-friendly,” low-trauma interface, which may lessen the possibility of any trauma associated with removable the system or components thereof. Additionally, the system allows for valuable system components, such as a negative-pressure device or unit, to be removed and reused in multiple iterations, without fear that the device may not be able to be reattached.

Some embodiments may relate to an apparatus for treating a tissue site with negative pressure. The apparatus may comprise a dressing, a dressing interface fluidly coupled to the dressing, and a conduit comprising a first end and a second end. The first end may be fluidly coupled to the dressing interface. The apparatus may also comprise a conduit chamber configured to retain at least a portion of the conduit and to allow the second end of the conduit to be extended from the dressing interface.

Additionally or alternatively, other embodiments may relate to a therapy unit for treating a tissue site with negative pressure. The therapy unit may comprise a reclosable fastener, a housing mounted to the reclosable fastener, the housing comprising a battery chamber, a pump chamber, and a port configured to receive a conduit. The therapy unit may also comprise a pump disposed in the pump chamber, the pump configured to be electrically coupled to a battery in the battery chamber and to deliver negative pressure to the port. The therapy unit may also comprise at least one cap disposed over the pump chamber and the battery chamber.

Additionally or alternatively, other embodiments may relate to a system for treating a tissue site with negative pressure, the system may comprise a dressing, a dressing interface fluidly coupled to the dressing, a therapy unit, and a conduit configured to couple the dressing interface to the therapy unit and to allow the therapy unit to be placed adjacent to the dressing within a range of about 40 millimeters to about 350 millimeters of the dressing interface.

Additionally or alternatively, other embodiments may relate to a method of treating a tissue site with negative pressure. The method may comprise providing a dressing and a conduit having a first end fluidly coupled to the dressing, applying the dressing to the tissue site, adhering a pump to an attachment surface adjacent to the tissue site, extending a second end of the conduit from the dressing to the pump, fluidly coupling the second end to the pump, and operating the pump to provide therapeutic levels of negative pressure to the tissue site through the dressing.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a perspective view of an example configuration of a dressing that may be associated with some embodiments of the therapy system of FIG. 1;

FIG. 3 is an assembly view illustrating additional details that may be associated with some example embodiments of the dressing of FIG. 2;

FIG. 4 is a sectional view taken along line 4-4 of FIG. 2 illustrating additional details that may be associated with some example embodiments of the dressing;

FIG. 5 is a perspective view illustrating additional details that may be associated with some example embodiments of the dressing of FIG. 2;

FIG. 6 is a perspective view illustrating additional details that may be associated with some example embodiments of the therapy system of FIG. 1;

FIG. 7 is an assembly view of an example configuration of a negative-pressure unit that may be associated with some embodiments of the therapy system of FIG. 1;

FIG. 8 is a perspective view illustrating additional details that may be associated with some example embodiments of the therapy system of FIG. 1;

FIGS. 9A-9D are perspective, partial cut-away views illustrating additional details that may be associated with some example embodiments of the therapy system of FIG. 1;

FIG. 10 is a sectional view taken along line 4-4 of FIG. 2 illustrating additional details that may be associated with some example embodiments of the therapy system of FIG. 1; and

FIG. 11 is a detail view of a portion of FIG. 10 illustrating additional details that may be associated with some example embodiments of the dressing.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

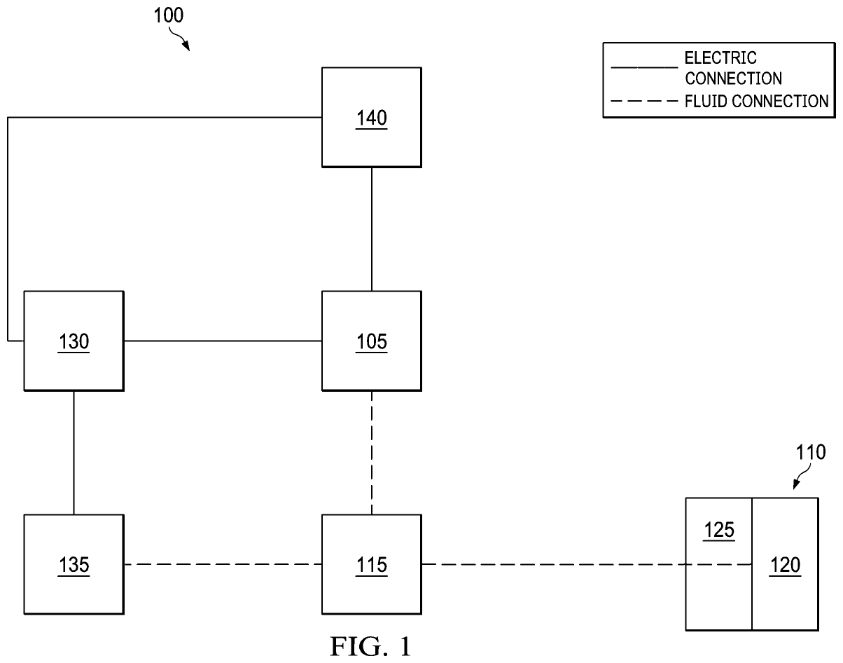

FIG. 1 is a simplified functional block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110, for example, a medical drape, may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user-interfaces that further facilitate therapy. For example, in some embodiments, the negative-pressure source 105 may be combined with the controller 130 or other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous materials such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inspire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudate and other fluid flows toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

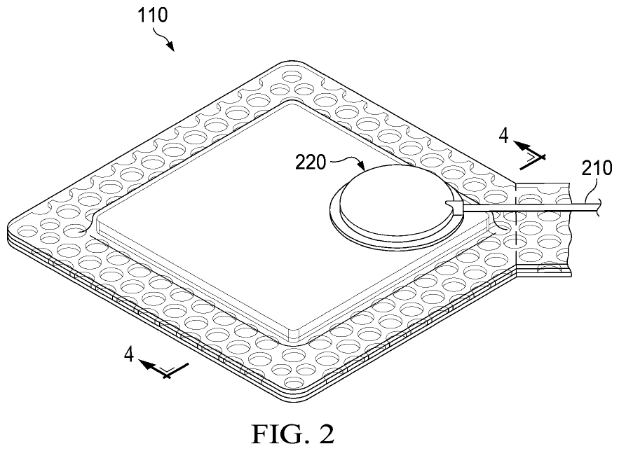

FIG. 2 is a perspective view of an example of the dressing 110 of FIG. 1, illustrating additional details that may be associated with the dressing 110. In the embodiment of FIG. 2, the dressing 110 includes a conduit 210 generally configured to convey a fluid between two ends of the conduit 210. The conduit 210 generally includes a first, proximal end fluidly coupled to the dressing 110, for example, via a dressing interface 220, and a second, distal end configured to be fluidly coupled to a negative-pressure source. The conduit 210 may have a length and diameter suitable for various applications.

The dressing 110 of FIG. 2 also includes a conduit chamber 220. The conduit chamber 220 is generally configured to retain at least a portion of the conduit 210 and to allow the distal end of the conduit 210 to be extended from the dressing 110, for example, dependent upon the placement of the dressing 110 with respect to the negative-pressure source to which the conduit 210 will be fluidly coupled.

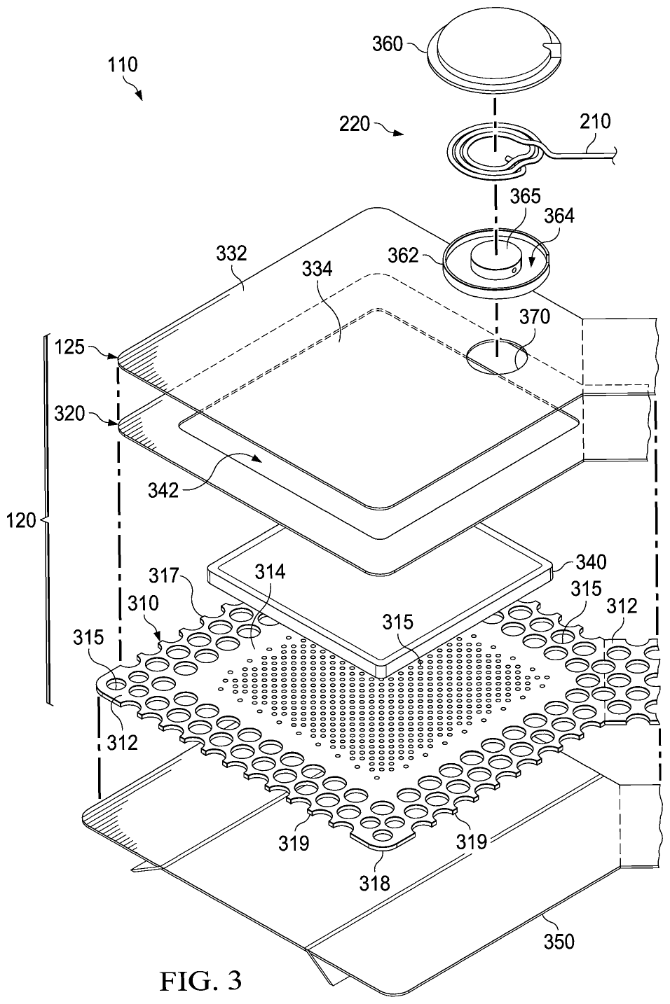

FIG. 3 is an assembly view of the dressing 110 of FIG. 2 illustrating additional details that may be associated with some embodiments in which the tissue interface 120 comprises more than one layer. In the embodiment of FIG. 3, the dressing 110 may be adapted to provide reduced pressure from the reduced-pressure source to a tissue site, and to store fluid extracted from the tissue site. The tissue interface 120 may include a contact layer 310, an adhesive 320, and a fluid distribution assembly 340. In various embodiments, components of the dressing 110, for example, the tissue interface 120, may be added or removed to suit a particular application.

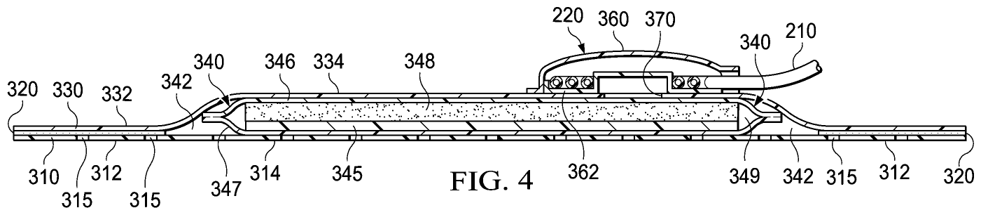

Referring to FIGS. 3 and 4, the contact layer 310 may have a periphery 312 surrounding a central portion 314, and a plurality of apertures 315 disposed through the periphery 312 and the central portion 314. The contact layer 310 may also have corners 318 and edges 319. The corners 318 and the edges 319 may be part of the periphery 312. One of the edges 319 may meet another of the edges 319 to define one of the corners 318. Further, the contact layer 310 may have a border 317 substantially surrounding the central portion 314 and positioned between the central portion 314 and the periphery 312.

In some embodiments, the central portion 314 of the contact layer 310 may be substantially square or rectangular in shape. The periphery 312 of the contact layer 310 may substantially uniformly surround the central portion 314 and may be substantially square or rectangular in shape. The periphery 312 of the contact layer 310 may substantially be bounded by the border 317. Although FIG. 3 depicts the central portion 314, the periphery 312, and the border 317, of the contact layer 310 as each having a substantially square or rectangular shape, in other embodiments, one or more of these may have any other shapes suitable to a particular application.

The apertures 315 in the contact layer 310 may have any shape, such as, for example, circles, squares, stars, ovals, polygons, slits, complex curves, rectilinear shapes, triangles, or other shapes. The apertures 315 may be formed by cutting, by application of local RF energy, or other suitable techniques for forming an opening. In the example of FIG. 3, each of the plurality of apertures 315 is substantially circular in shape, having a diameter and an area. The area of each of the plurality of apertures 315 may refer to an open space or open area defining each of the apertures 315. The diameter of each of the apertures 315 may define the area of each of the apertures 315, for example, the area of a particular of the plurality of apertures 315 may be defined by multiplying the square of half the diameter (that is, the radius) of that particular aperture by the value 3.14. Thus, the following equation may define the area of one of the apertures 315: Area=3.14*(diameter/2)\hat{ }2. The area of the plurality the apertures 315 described in the illustrative embodiments herein may be substantially similar to the area in other embodiments (not shown) for the plurality of apertures 315 that may have non-circular shapes. The diameter of each of the apertures 315 may be substantially the same, or each of the diameters may vary depending, for example, on the position of one of the apertures 315 in the contact layer 310. For example, in the embodiment of FIG. 3, the diameter of the apertures 315 in the periphery 312 of the contact layer 310 may be larger than the diameter of the apertures 315 in the central portion 314 of the contact layer 310. Further, the diameter of each of the apertures 315 may be about 1 millimeter to about 50 millimeters. In some embodiments, the diameter of each of the apertures 315 may be about 1 millimeter to about 20 millimeters. The apertures 315 may have a uniform pattern or may be randomly distributed on the contact layer 310. In some embodiments, the size and configuration of the apertures 315 may be configured to control the adherence of the dressing 110 to the epidermis, as described below. In some embodiments, the apertures 315 disposed in the periphery 312 may have a diameter between about 9.8 millimeters to about 10.2 millimeters. Additionally or alternatively, in some embodiments, the apertures 315 disposed in the central portion 314 may have a diameter between about 1.8 millimeters to about 2.2 millimeters.

The contact layer 310 may comprise a soft, pliable material suitable for providing a fluid seal with a tissue site, as described herein. For example, in various embodiments the contact layer 310 may comprise a silicone, a silicone gel, a soft silicone, hydrocolloid, hydrogel, polyurethane gel, polyolefin gel, hydrogenated styrenic copolymer gels, a foamed gel, a soft closed cell foam such as polyurethanes and polyolefins coated with an adhesive described below, polyurethane, polyolefin, or hydrogenated styrenic copolymers. The contact layer 310 may have a thickness between about 500 microns (μm) and about 1000 microns (μm). In some embodiments, the contact layer 310 may have a stiffness between about 5 Shore 00 and about 80 Shore 00. The contact layer 310 may be comprised of hydrophobic or hydrophilic materials.

In some embodiments, the contact layer 310 may be a hydrophobic-coated material. For example, the contact layer 310 may be formed by coating a spaced material, such as, for example, woven, nonwoven, molded, or extruded mesh with a hydrophobic material. The hydrophobic material for the coating may be a soft silicone, for example. In this manner, the adhesive 320 may extend through openings in the spaced material analogous to the apertures 315 described below.

At least one of the apertures 315 in the periphery 312 of the contact layer 310 may be positioned at the edges 319 of the periphery 312 and may have an interior cut open or exposed at the edges 319 that is in fluid communication in a lateral direction with the edges 319. The lateral direction may refer to a direction toward the edges 319 and in the same plane as the contact layer 310. As shown in FIG. 3, a plurality of the apertures 315 in the periphery 312 may be positioned proximate to or at the edges 319 and in fluid communication in a lateral direction with the edges 319. The apertures 315 disposed proximate to or at the edges 319 may be spaced substantially equidistant around the periphery 312. However, in some embodiments, the spacing of the apertures 315 disposed proximate to or at the edges 319 may be irregular.

The adhesive 320 may be a medically-acceptable adhesive. Additionally, in some embodiments the adhesive 320 may be characterized as flowable. For example, the adhesive 320 may comprise an acrylic adhesive, rubber adhesive, high-tack silicone adhesive, polyurethane, or other adhesive substance. In some embodiments, the adhesive 320 may be a pressure-sensitive adhesive comprising an acrylic adhesive with coating weight of 15 grams/m\hat{ }2 (gsm) to 70 grams/m\hat{ } 2 (gsm). The adhesive 320 may be a layer having substantially the same shape as the periphery 312 of the contact layer 310, for example, as shown in FIG. 3. In various embodiments, the layer of the adhesive 320 may be continuous or discontinuous. For example, in some embodiments, discontinuities in the adhesive 320 may be provided by apertures in the adhesive 320. Apertures in the adhesive 320 may be formed after application of the adhesive 320 or by coating the adhesive 320 in patterns on a carrier layer, such as, for example, a side of the cover 125. Further, discontinuities in the adhesive 320 may be sized to control the amount of the adhesive 320 extending through the apertures 315 in the contact layer 310 to reach the epidermis when the dressing is placed with respect to a tissue site. Additionally, discontinuities in the adhesive 320 may also be sized to enhance the Moisture Vapor Transfer Rate (MVTR) of the dressing 110.

Various factors that may control the adhesion strength of the dressing 110 may include the diameter and number of the apertures 315 in the contact layer 310, the thickness of the contact layer 310, the thickness and amount of the adhesive 320, and the tackiness of the adhesive 320. For example, an increase in the amount of the adhesive 320 extending through the apertures 315 generally corresponds to an increase in the adhesion strength of the dressing 110. Also for example, a decrease in the thickness of the contact layer 310 generally corresponds to an increase in the amount of adhesive 320 extending through the apertures 315. Thus, the diameter and configuration of the apertures 315, the thickness of the contact layer 310, and the amount and tackiness of the adhesive utilized may be varied to provide a desired adhesion strength for the dressing 110. For example, the thickness of the contact layer 310 may be about 200 microns, the adhesive 320 may have a thickness of about 30 microns and a tackiness of 2000 grams per 25 centimeter wide strip, and the diameter of the apertures in the contact layer 310 may be about 10 millimeters.

In some embodiments, the tackiness of the adhesive 320 may vary in different locations of the contact layer 310. For example, in locations of the contact layer 310 where the apertures 315 are comparatively large, such as the apertures 315 within the periphery 312, the adhesive 320 may have a lower tackiness than other locations of the contact layer 310 where the apertures 315 are smaller, such as the apertures 315 in the central portion 314. In this manner, locations of the contact layer 310 having larger apertures 315 and lower tackiness adhesive 320 may have an adhesion strength comparable to locations having smaller apertures 315 and higher tackiness adhesive 320.

Clinical studies have shown that the configuration described herein for the contact layer 310 and the adhesive 320 may reduce the occurrence of blistering, erythema, and leakage when in use. Additionally, such a configuration may provide, for example, increased patient comfort and increased durability of the dressing 110.

Also referring to the embodiment of FIG. 3, a release liner 350 may be attached to or positioned adjacent to the contact layer 310, for example, to protect the adhesive 320 prior to application of the dressing 110 to a tissue site. Prior to application of the dressing 110 to the tissue site, the contact layer 310 may be positioned between the cover 125 and the release liner 350. Removal of the release liner 350 may expose the contact layer 310 and the adhesive 320 for application of the dressing 110 to the tissue site. The release liner 350 may also provide stiffness to assist with, for example, deployment of the dressing 110. The release liner 350 may be, for example, a casting paper, a film, or polyethylene. Further, the release liner 350 may be a polyester material such as polyethylene terephthalate (PET), or similar polar semi-crystalline polymer. The use of a polar semi-crystalline polymer for the release liner 350 may substantially preclude wrinkling or other deformation of the dressing 110. For example, the polar semi-crystalline polymer may be highly orientated and resistant to softening, swelling, or other deformation that may occur when brought into contact with components of the dressing 110, or when subjected to temperature or environmental variations, or sterilization. Further, a release agent may be disposed on a side of the release liner 350 that is configured to contact the contact layer 310. For example, the release agent may be a silicone coating and may have a release factor suitable to facilitate removal of the release liner 350 by hand and without damaging or deforming the dressing 110. In some embodiments, the release agent may be flourosilicone. In other embodiments, the release liner 350 may be uncoated or otherwise used without a release agent.

In some embodiments, the cover 125 has a periphery 332 and a central portion 334. The cover 125 may additionally include a dressing cover aperture 370 disposed through the cover 125. Further, the cover 125 may be configured to cover at least a portion of the contact layer 310. For example, the periphery 332 of the cover 125 may be positioned proximate to the periphery 312 of the contact layer 310 such that the central portion 334 of the cover 125 and the central portion 314 of the contact layer 310 define an enclosure 342. The adhesive 320 may be positioned at least between the periphery 332 of the cover 125 and the periphery 312 of the contact layer 310. The cover 125 may cover the contact layer 310.

In some embodiments, the cover 125 may include the conduit chamber 220, for example, the conduit chamber 220 may be disposed on or incorporated into the cover 125. The conduit chamber 220 may generally define a chamber in which at least a portion of the conduit 210 is retained. For example, in the embodiment of FIG. 3, the conduit chamber 220 comprises a conduit chamber base 362 and a conduit chamber cover 360 that cooperatively define a conduit space 364.

The conduit chamber base 362 may include a base portion and a wall portion.

The conduit chamber base 362 may have any suitable shape, for example, dependent upon the desired shaped of the conduit space 364 and the desired disposition of the portion of the conduit 210 within the conduit space 364. For example, in the embodiment of FIG. 3 the conduit chamber base 362 is generally circular, although in other embodiments a conduit chamber base may be rectangular, square, oblong, or any other suitable shape.

Also in the embodiment of FIGS. 3-4, the conduit chamber 220 comprises a conduit interface 365 which may be positioned proximate to the cover 125 and in fluid communication with the enclosure 342 of the dressing 110 through the dressing cover aperture 370 in the cover 125, for example, to provide reduced pressure from the negative-pressure source 105 to the dressing 110.

In some embodiments, the conduit chamber base 362, the conduit chamber cover 360, the conduit interface 365, or combinations of these may comprise a medical-grade, soft polymer or other pliable material. As non-limiting examples, the conduit chamber base 362, the conduit chamber cover 360, and/or the conduit interface 365 may be formed from polyurethane, polyethylene, polyvinyl chloride (PVC), fluorosilicone, or ethylene-propylene, etc. In some illustrative, non-limiting embodiments, the conduit chamber base 362, the conduit chamber cover 360, and/or conduit interface 365 may be molded from DEHP-free PVC. The conduit chamber base 362, the conduit chamber cover 360, and/or conduit interface 365 may be formed in any suitable manner such as by molding, casting, machining, or extruding. Further, the conduit chamber base 362, the conduit chamber cover 360, and/or the conduit interface 365 may be formed as an integral unit or as individual components and may be coupled together and/or to the dressing 110 by, for example, an adhesive or welding.

In some embodiments, the conduit 210 may be coupled to and in fluid communication between the negative-pressure source 105 and the dressing 110. For example, the conduit 210 may have an internal lumen may have a diameter between about 0.5 millimeters to about 3.0 millimeters. More specifically, the diameter of the internal lumen may be about 1 millimeter to about 2 millimeters. The conduit interface 365 may be fluidly coupled to the conduit 210 in any suitable manner, such as, for example, by an adhesive, solvent or non-solvent bonding, welding, or interference fit. The conduit interface 365 may be coupled in fluid communication with the dressing 110.

The conduit 210 may be, for example, a flexible polymer tube. The conduit 210 may have any suitable length, dependent upon its intended utility. For example, in some embodiments the conduit 210 may have a length from about 100 mm to about 500 mm, or from about 150 mm to about 400 mm, or from about 250 to about 350 mm. The distal end of the conduit 210 may include a coupling for attachment to the negative-pressure source 105. Additionally, the conduit 210 may be adapted to connect between the conduit 210 and the dressing 110 for providing fluid communication with the negative-pressure source 105. For example, the distal end of the conduit 210 may include a suitable connect, such as an interference-fit or compression-fit connector. In some embodiments, the conduit 210 may also include one or more filters, for example, incorporated within the connector at the distal end of the conduit 210, configured to block liquid passages. Examples of such filters include sintered polymer gel-blocking filters and various hydrophobic filters.

The conduit 210 may be disposed in the conduit chamber 220 such that conduit 210 can be extended from the conduit chamber 220 upon the application of a force to pull the conduit 210 from the conduit chamber 220. In some embodiments, the conduit 210 may be held in place within the conduit chamber 220 via the application of pressure from the conduit chamber cover 360. For example, the conduit 210 may held within the conduit chamber 220 via compression between the conduit chamber base 362 and the conduit chamber cover 360. Additionally or alternatively, the conduit 210 may held within the conduit chamber 220 via an adhesive applied to the conduit chamber base 362 and/or the conduit chamber cover 360. The force necessary to extend the conduit 210 away from the conduit chamber 220 may be such that a user can easily pull that conduit 210 from the conduit chamber 220; for example, the necessary force may be less than about 2N, or less than about 1.5N, or from about 0.5N to about 2N, or from about 1N to about 2N.

The conduit 210 may be disposed within the conduit chamber 220 in any suitable pattern, preferably, in a pattern that will allow the conduit 210 to be extended from the conduit chamber 220 while resisting any kinking or bending that might cause fluid movement via the internal lumen to be obstructed. For example, in the embodiment of FIG. 3, the conduit 210 is disposed in the conduit chamber 220 in a coiled pattern. For example, the conduit 210 is coiled around the conduit interface 365 such that the conduit 210 uncoils as it is extended from the conduit chamber 220. In other embodiments, the conduit 210 is disposed in the conduit chamber 220 in a serpentine, back-and-forth, or sinusoidal pattern.

In some embodiments, the fluid distribution assembly 340 is configured to collect or distribute fluid across to a tissue site to which the dressing 110 applied. For example, the fluid distribution assembly 340 may comprise a manifold, for example, a reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the reticulated foam may also vary according to needs of a prescribed therapy. The 25% compression load deflection of the fluid distribution assembly 340 may be at least 0.35 pounds per square inch, and the 65% compression load deflection may be at least 0.43 pounds per square inch. In some embodiments, the tensile strength of the fluid distribution assembly 340 may be at least 10 pounds per square inch. The fluid distribution assembly 340 may have a tear strength of at least 2.5 pounds per inch. In some embodiments, the fluid distribution assembly 340 may be foam comprised of polyols such as polyester or polyether, isocyanate such as toluene diisocyanate, and polymerization modifiers such as amines and tin compounds. In some examples, the fluid distribution assembly 340 may be reticulated polyurethane foam such as found in the V.A.C.® GRANUFOAM™ Dressing or V.A.C.® VERAFLO™ dressing, both available from Kinetic Concepts, Inc. of San Antonio, Texas.

Additionally or alternatively, the fluid distribution assembly 340 may be either hydrophobic or hydrophilic. In an example in which the fluid distribution assembly 340 may be hydrophilic, the fluid distribution assembly 340 may also wick fluid away from a tissue site, while continuing to distribute negative pressure to the tissue site. The wicking properties of the fluid distribution assembly 340 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the fluid distribution assembly 340 may include or be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The fluid distribution assembly 340 may further serve as a scaffold for new cell-growth, or a scaffold material may be used in conjunction with the fluid distribution assembly 340 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

Additionally or alternatively, in some embodiments the fluid distribution assembly 340 may be configured to manage or control fluid movement via the dressing 110. For example, referring to FIG. 4, a cross-sectional view of the dressing 110 of FIGS. 2 and 3 is shown, illustrating additional aspects of the dressing. In the embodiment of FIG. 4, the fluid distribution assembly 340 may be disposed within the enclosure 342 positioned between the contact layer 310 and the cover 125. Also, the fluid distribution assembly 340 may include one or more wicking layers. For example, the fluid distribution assembly 340 may include a first wicking layer 346 and a second wicking layer 347.

Further, in some embodiments, the fluid distribution assembly 340 may include an absorbent material such as an absorbent layer 348. Although the absorbent material is depicted in the form of a layer as the absorbent layer 348, in some embodiments, the absorbent material may have a granular form or other suitable form. The absorbent layer 348 may be positioned in fluid communication between the first wicking layer 346 and the second wicking layer 347. The first wicking layer 346 may have a grain structure adapted to wick fluid along a surface of the first wicking layer 346. Similarly, the second wicking layer 347 may have a grain structure adapted to wick fluid along a surface of the second wicking layer 347. For example, the first wicking layer 346 and the second wicking layer 347 may wick or otherwise transport fluid in a lateral direction along the surfaces of the first wicking layer 346 and the second wicking layer 347, respectively. The surfaces of the first wicking layer 346 and the second wicking layer 347 may be normal relative to the thickness of each of the first wicking layer 346 and the second wicking layer 347. The wicking of fluid along the first wicking layer 346 and the second wicking layer 347 may enhance the distribution of the fluid over a surface area of the absorbent layer 348 that may increase absorbent efficiency and resist fluid blockages. Fluid blockages may be caused by, for example, fluid pooling in a particular location in the absorbent layer 348 rather than being distributed more uniformly across the absorbent layer 348. The laminate combination of the first wicking layer 346, the second wicking layer 347, and the absorbent layer 348 may be adapted as described herein to maintain an open structure, resistant to blockage, and capable of maintaining fluid communication there through.

In some embodiments, a peripheral portion of the first wicking layer 346 may be coupled to a peripheral portion of the second wicking layer 347 by a bond to define a wicking enclosure 349 between the first wicking layer 346 and the second wicking layer 347. In some exemplary embodiments, the wicking enclosure 349 may surround or otherwise encapsulate the absorbent layer 348 between the first wicking layer 346 and the second wicking layer 347. In some embodiments, a single wicking layer may surround the absorbent layer 348 to form the wicking enclosure 349. Accordingly, the absorbent material or the absorbent layer 348 may be surrounded by at least one wicking layer. Further, a portion of the wicking layer may be coupled to another portion of the wicking layer to form the wicking enclosure 349.

Additionally or alternatively, in some embodiments, the fluid distribution assembly 340 may include, without limitation, any number of fluid transmission layers, wicking layers, or absorbent layers as desired for treating a particular tissue site. In some embodiments, at least one wicking layer may surround the absorbent layer 348. In some embodiments, the absorbent layer 348 may be printed on, carried, or otherwise supported by the at least one wicking layer. In such an embodiment, the at least one wicking layer may not surround the absorbent layer 348. Further, in some embodiments, at least one intermediate wicking layer may be disposed in fluid communication between the absorbent layer 348 and the second wicking layer 347. In such an embodiment, the second wicking layer 347 may be positioned between the intermediate wicking layer and the cover 125. Further, including additional absorbent layers 348 may increase the absorbent mass of the fluid distribution assembly 340 and generally provide greater fluid capacity. However, for a given absorbent mass, multiple light coat-weight absorbent layers 348 may be utilized rather than a single heavy coat-weight absorbent layer 348 to provide a greater absorbent surface area for further enhancing the absorbent efficiency.

In some embodiments, one or more of the wicking layers, such as the first and second wicking layers 346, 347, may include a fluid distribution side and a fluid acquisition side. The fluid distribution side may be positioned facing an opposite direction from the fluid acquisition side. The fluid distribution side may include longitudinal fibers that define a grain structure, which may be oriented substantially in a longitudinal direction along a length of the wicking layers. The fluid acquisition side may include vertical fibers. The vertical fibers may be oriented substantially vertical or normal relative to the longitudinal fibers and the length of wicking layers. In some embodiments, the fluid acquisition side of both the second wicking layer 347 may be positioned facing the absorbent layer 348, and the fluid acquisition side of the first wicking layer 346 may be positioned facing away from the absorbent layer 348.

In some embodiments, the absorbent layer 348 may be a hydrophilic material adapted to absorb fluid from, for example, a tissue site. Materials suitable for the absorbent layer 348 may include Luquafleece® material, Texsus FP2326, BASF 402C, Technical Absorbents 2317 available from Technical Absorbents (www.techabsorbents.com), sodium polyacrylate super absorbers, cellulosics (carboxy methyl cellulose and salts such as sodium CMC), or alginates. Materials suitable for the first wicking layer 346 and the second wicking layer 347 may include any material having a grain structure capable of wicking fluid as described herein, such as, for example, Libeltex TDL2 80 gsm.

In various embodiments, the fluid distribution assembly 340 may comprise a pre-laminated structure manufactured at a single location or individual layers of material stacked upon one another as described above. Individual layers of the fluid distribution assembly 340 may be bonded or otherwise secured to one another without adversely affecting fluid management by, for example, utilizing a solvent or non-solvent adhesive, or by thermal welding.

In some embodiments, the enclosure 342 defined by the contact layer 310 and the cover 125 may include an anti-microbial layer 345. The addition of the anti-microbial layer 345 may reduce the probability of excessive bacterial growth within the dressing 110 to permit the dressing 110 to remain in place for an extended period. The anti-microbial layer 345 may be, for example, an additional layer included as a part of the fluid distribution assembly 340 as depicted in FIG. 4, or a coating of an anti-microbial agent disposed in any suitable location within the dressing 110. The anti-microbial layer 345 may be comprised of elemental silver or similar compound, for example. In some embodiments, the anti-microbial agent may be formulated in any suitable manner into other components of the dressing 110.

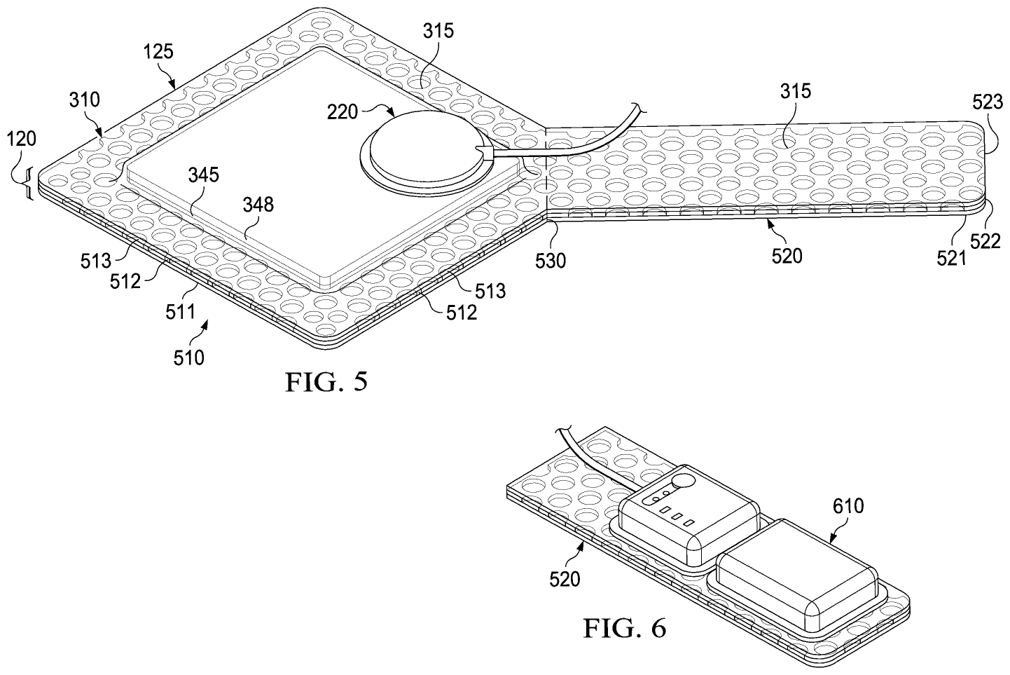

In one or more of the figures that follow, one or more layers of the dressing 110 are shown as transparent in order to illustrate various aspects of the dressing 110, though a layer may be some degree of opaque, translucent, or transparent in various embodiments. FIG. 5 is a perspective view illustrating additional details that may be associated with some example embodiments of the dressing of FIG. 2. Referring to FIG. 5, in some embodiments one or more layers of the tissue interface 120 can include both a dressing portion 510 and tail portion 520. For example, in the embodiment of FIG. 5, the contact layer 310 comprises a contact layer dressing portion 511 and a contact layer tail portion 521, the adhesive 320 comprises an adhesive dressing portion 512 and an adhesive tail portion 522, and the cover 125 comprises a dressing cover dressing portion 513 and a dressing cover tail portion 523.

The dressing portion 510 may take any suitable shape or size, for example, dependent upon the intended utility. For example, while the embodiment of FIG. 5 illustrates the dressing portion 510 as generally square-shaped or diamond-shaped, in other embodiments a dressing portion may be rectangular, circular, elliptical, ovoid, or another suitable shape, for example. Also, the tail portion 520 may be shaped and sized to meet an intended utility.

In some embodiments, the dressing portion 510 may be separable with respect to the tail portion 520. For example, the dressing portion 510 may be separated from the tail portion 520 such as by cutting, for example, at a cut-line 530. Additionally or alternatively, in some embodiments the cut-line 530 can include perforations or other lines of weakness allowing the dressing portion 510 to be separated from the tail portion 520 such as by tearing or pulling.

FIG. 6 is a perspective view illustrating additional details that may be associated with some example embodiments of the therapy system of FIG. 1. Referring to the embodiment of FIG. 6, a negative-pressure unit 610 is illustrated attached to the tail portion 520, for example, attached to the dressing cover tail portion 523. In some embodiments, the tail portion 520 may be configured for attachment of the negative-pressure source 105. In some embodiments, the negative-pressure unit 610 may be configured as a stand-alone unit, for example, capable of providing negative pressure to the dressing 110 without need of a physical connection to any other component. For example, the negative-pressure unit 610 may be characterized as portable or mobile. Additionally, the negative-pressure unit 610 may be characterized as a wearable device. For example, the negative-pressure unit 610 may be relatively small and compact in size, enabling a user to carry the negative-pressure unit 610 and/or for the negative-pressure unit 610 to be affixed to the user.

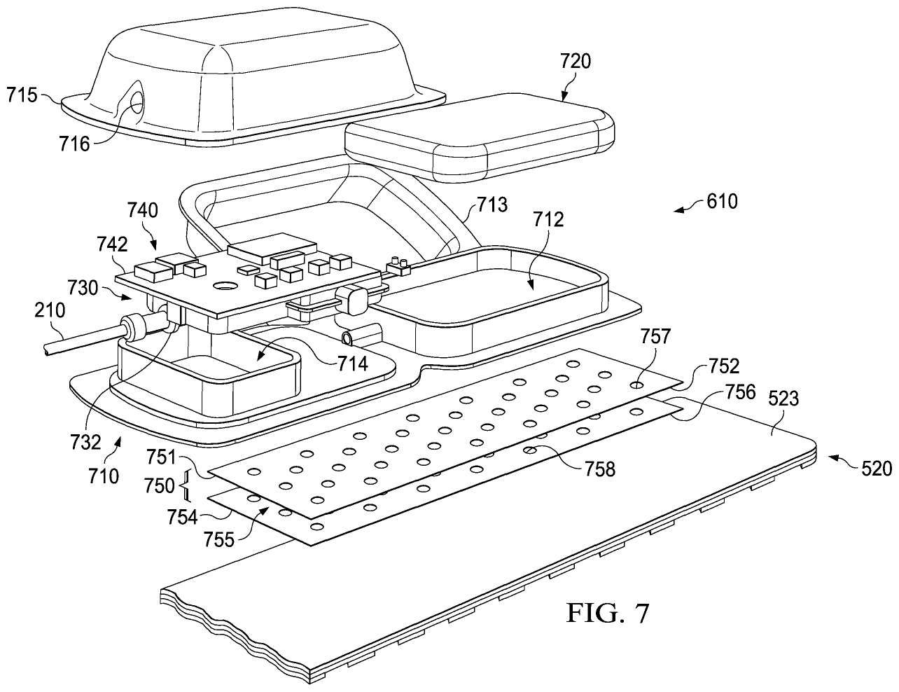

FIG. 7 is an assembly view of an example configuration of a negative-pressure unit that may be associated with some embodiments of the therapy system of FIG. 1. Referring to FIG. 7, an assembly view of an example of the negative-pressure unit 610 is illustrated. In the embodiment of FIG. 7, the negative-pressure unit 610 generally comprises a housing 710; a power unit, such as a battery; and a pump 730.

The housing 710 may comprise a battery chamber 712 and a pump chamber 714.

The housing 710 may also include one or more caps configured to enclose and/or seal the battery chamber 712 and the pump chamber 714. For example, in the embodiment of FIG. 7 the housing 710 includes a battery chamber cap 713 and a pump chamber cap 715. The battery chamber 712 and battery chamber cap 713 may cooperatively retain and seal the battery 720 within the housing 710. The battery 720 may be removable from the housing 710, for example, to enable the battery to be recharged or replaced. Likewise, the pump chamber 714 and pump chamber cap 715 may cooperatively retain and seal the pump 730 within the housing 710. When the battery 720 inserted within the battery chamber 712, the housing 710 may provide electrical and signal communication to the pump 730, for example, to enable operation of the pump 730. The pump chamber 714 and/or pump chamber cap 715 may include a passage 716 enabling the conduit 210 to be fluidly coupled to a fluid port of the pump 730.

Also, in some embodiments the negative-pressure unit 610 may include a user-interface 740, for example, configured to enable a user to control and/or monitor the operation of the negative-pressure unit 610. In various embodiments, the user-interface 740 may include any suitable combination of push-buttons, touch-pads, switches, dials, or the like. Additionally, the user-interface 740 may also include one or more visual, auditory, or tactile notification devices, for example, lights or alarms, configured to alert the user to various operational states associated with the negative-pressure unit 610. In the embodiment of FIG. 7, the user-interface 740 may be disposed on or integrated into the pump 730, for example, as part of a printed-circuit board 742, which may also include one or more modules configured to control the operation of the pump 730 or a component thereof.

In the embodiment of FIG. 7, the negative-pressure unit 610 may be attached to the tail portion 520, for example, the dressing cover tail portion 523, via a reclosable fastener 750. As used herein, the reclosable fastener 750 refers to a fastener or fastener system capable of being opened and closed, for example, engaged and disengaged, multiple times with little or no reduction in the fastening capacity of the reclosable fastener 750.

In some embodiments, the reclosable fastener 750 may include two or more layers. For example, in the embodiment of FIG. 7, the reclosable fastener 750 may include a first layer 751 and a second layer 754. Each of the first layer 751 and the second layer 754 may include reclosable surface. For example, the first layer 751 and the second layer 754 may include a first reclosable surface 752 and a second reclosable surface 755, respectively. Generally, the first reclosable surface 752 and second reclosable surface 755 may be configured to interact so as to allow the reclosable fastener 750 to be opened or closed multiple times for example, engaged and disengaged, multiple times with little or no reduction in the interaction between the respective surfaces.

For example, in some embodiments each of the first reclosable surface 752 and the second reclosable surface 755 may include an array of stems protruding therefrom. The array of stems of the first reclosable surface 752 may be engaged with and/or disengaged from the array of stems of the second reclosable surface 755. When closed or engaged, the arrays of stems of the two layers may interact to hold the first layer 751 and second layer 754 in place with respect to each other. An example of this configuration of reclosable fastener is commercially-available as Dual Lock™ from 3M™ in Maplewood, Minnesota.

Additionally or alternatively, in some embodiments one of the first reclosable surface 752 and the second reclosable surface 755 may include an array of hooks protruding therefrom and the other of the first reclosable surface 752 and the second reclosable surface 755 may include an array of loops. The array of hooks may be engaged with and/or disengaged from the array of loops of the other layer and, when closed or engaged, the arrays of hooks and loops of the two layers may interact to hold the first layer 751 and second layer 754 in place with respect to each other. An example of this configuration of reclosable fastener is commercially-available as Velcro, from a variety of manufacturers.

Additionally or alternatively, in some embodiments one or both of the first reclosable surface 752 and the second reclosable surface 755 may include an adhesive or a component of an adhesive system, such as a pressure-sensitive adhesive. For example, in some embodiments the adhesive of the one of the first reclosable surface 752 and the second reclosable surface 755 may be engaged with and/or disengaged from the reclosable surface of the other layer and, when closed or engaged, the adhesive may interact the first reclosable surface 752 and the second reclosable surface 755 to hold the first layer 751 and second layer 754 in place with respect to each other. Additionally or alternatively, in some embodiments, a first component of an adhesive system may be disposed on one of the one of the first reclosable surface 752 and the second reclosable surface 755 and a second component of the adhesive system may be disposed on the other the first reclosable surface 752 and the second reclosable surface 755. The first reclosable surface 752 and the second reclosable surface 755 may be engaged with and/or disengaged from the each other such that, when closed or engaged, the first and second components of the adhesive system may interact the first reclosable surface 752 and the second reclosable surface 755 to hold the first layer 751 and second layer 754 in place with respect to each other.

Also, in some embodiments each of the first layer 751 and the second layer 754 may each include a fixed surface. The fixed surface may generally be configured to allow the first layer 751 or the second layer 754 to be affixed, for example, permanently or semi-permanently, to a substrate. For example, the first layer 751 and the second layer 754 may include a first fixed surface 753 and a second fixed surface 756, respectively. Generally, the first fixed surface 753 and second fixed surface 756 may be configured to hold the first layer 751 and the second layer 754 in place with respect to two substrates joined by the reclosable fastener 750 such that the application of a force to separate those substrates causes the first reclosable surface 752 and the second reclosable surface 755 to be opened or disengaged while the first fixed surface 753 and second fixed surface 756 remain affixed to the respective substrates. For example, in some embodiments, the reclosable fastener 750 may be characterized as exhibiting a detachment force, referring to the force necessary to open or disengage the reclosable fastener 750, of less than ION, for example, less than about 6N, or less than about 5N, or from about 2N to about 8 N, or from about 3N to about 6N, or from about 4N to about 5N. For example, the first reclosable surface 752 and the second reclosable surface 755 may be characterized as exhibiting a detachment force, referring to the force necessary the first reclosable surface 752 and the second reclosable surface 755, of less than ION, for example, less than about 6N, or less than about 5N, or from about 2N to about 8 N, or from about 3N to about 6N, or from about 4N to about 5N. Additionally or alternatively, the first fixed surface 753 and second fixed surface 756 may each be characterized as exhibiting a detachment force, referring to the force necessary to separate the first fixed surface 753 or second fixed surface 756 from a respective substrate, of at least ION, or at least 12N, or at least 14N, or at least 16N, or at least 18N, or at least 20.

In some embodiments, reclosable fastener 750 may be perforated. For example, the first layer 751, the second layer 754, or both may be perforated. For example, in the embodiment of FIG. 7, the first layer 751 includes an array of perforations 757 and the second layer 754 also includes an array of perforations 758. The arrays of perforations 757, 758 may be disposed in the first layer 751 and the second layer 754, respectively, such that, when the first layer 751 and the second layer 754 are engaged, the perforations 757 in the first layer 751 may be at least partially aligned with the perforations 758 in the second layer 754. In some embodiments, the presence of the perforations 757 in the first layer 751 and/or the perforations 758 in the second layer 754 may be effective to modulate or tailor the detachment force necessary to separate the first reclosable surface 752 and the second reclosable surface 755. Additionally or alternatively, in some embodiments, the presence of the perforations 757 in the first layer 751 and/or the perforations 758 in the second layer 754 may be effective to maintain moisture-vapor transmission through the reclosable fastener 750, for example, to modulate the MVTR via the first layer 751, the second layer 754, or both. For example, the perforations 757 in the first layer 751 and/or the perforations 758 in the second layer 754 may be effective to yield a MVTR of at least 250 grams per square meter per twenty-four hours via the tail portion 520.

In the embodiment of FIG. 7, the first layer 751 is affixed to the housing 710 via the first fixed surface 753 and the second layer 754 is affixed to the tail portion 520, particularly, to the dressing cover tail portion 523, via the second fixed surface 756. Alternatively, in some embodiments, the first layer 751 is affixed to the tail portion 520, particularly, to the dressing cover tail portion 523, via the first fixed surface 753 and the second layer 754 is affixed to the housing 710 via the second fixed surface 756.

Additionally or alternatively, in some embodiments a reclosable fastener may include a single layer. For example, in such an embodiment the reclosable fastener may include each of a fixed surface and a reclosable surface comprising an adhesive. The adhesive of the fixed surface may exhibit a greater detachment force than the adhesive of the reclosable surface. For example, such that the fixed surface remains fixed with respect to its respective substrate. For example, the fixed surface may remain fixed to the tail portion 520, particularly, to the dressing cover tail portion 523, while the reclosable surface may be preferentially separated from its respective substrate.

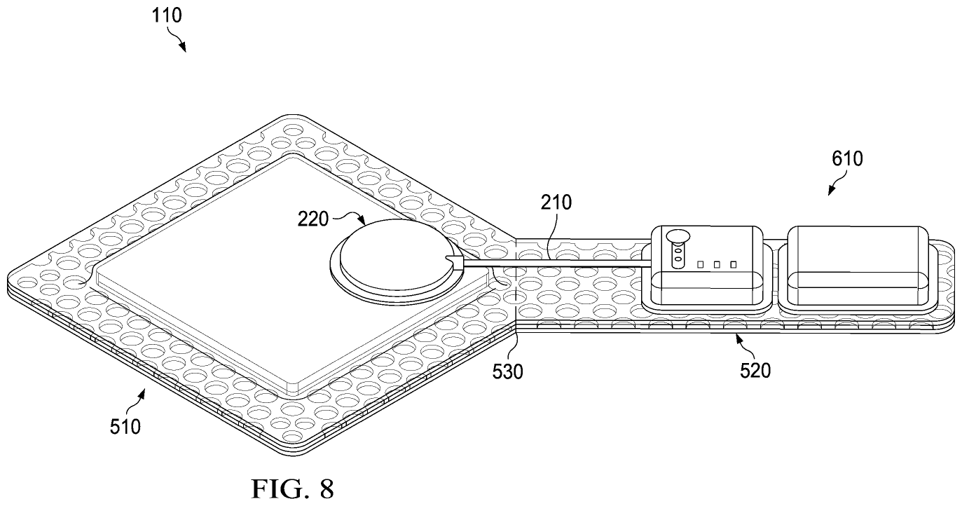

FIG. 8 is a perspective view illustrating additional details that may be associated with some example embodiments of the therapy system of FIG. 1. In some embodiments, the capability to attach the negative-pressure unit 610, via the reclosable fastener 750, to the tail portion 520 may allow the negative-pressure unit 610 to be worn by a user, for example, a patient receiving negative-pressure therapy. For example, referring to FIG. 8, the negative-pressure unit 610 is shown attached to the tail portion 520. As such, by affixing the dressing 110 to a user, the dressing 110 may function as a base to which the negative-pressure unit 610 can be attached, allowing the negative-pressure unit 610 to be worn by the user. Also in the embodiment of FIG. 8, the negative-pressure unit 610 is shown fluidly-coupled via the conduit 210 to the dressing 110, for example, to provide negative pressure to the dressing 110, for example, to the enclosure 342.

Additionally, in the implementation illustrated by FIG. 8 the dressing portion 510 and tail portion 520 are shown remaining attached, thereby providing the negative-pressure unit 610 in close proximity to, for example, adjacent to, the dressing portion 510. In other implementations, however, it may desirable to position the negative-pressure unit 610 less proximate to the dressing portion 510. For example, in various implementations and dependent upon the location of the tissue site(s) receiving negative-pressure therapy, it may be desirable to position the negative-pressure unit 610 adjacent to the dressing portion 510 as show in FIG. 8 or at various locations spaced apart from the dressing portion 510.

In some embodiments where it is desirable to position the negative-pressure unit 610 apart from the dressing portion 510, the tail portion 520 can also be used to enable the user to attach the negative-pressure unit 610 apart from the dressing portion 510. For example, the dressing portion 510 may be separated from the tail portion 520, for example, by cutting or tearing at the cut-line 530. With the dressing the dressing portion 510 separated from the tail portion 520, the dressing portion 510 may be applied to or positioned with respect to a tissue site for therapy and the tail portion 520 may be affixed to the user separately, for example, such that the negative-pressure unit 610 may be affixed to the tail portion 520 at a position apart from the dressing portion 510.

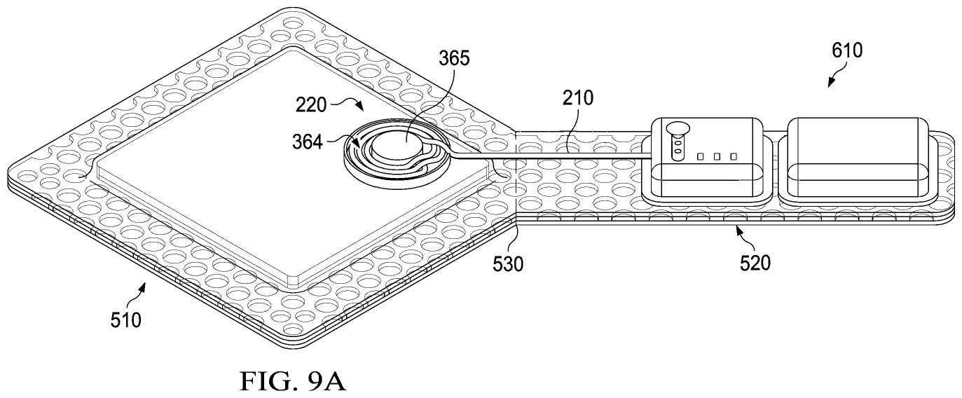

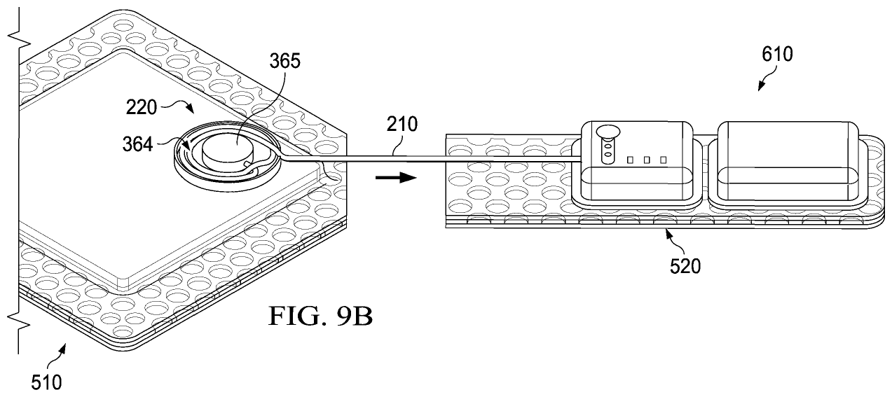

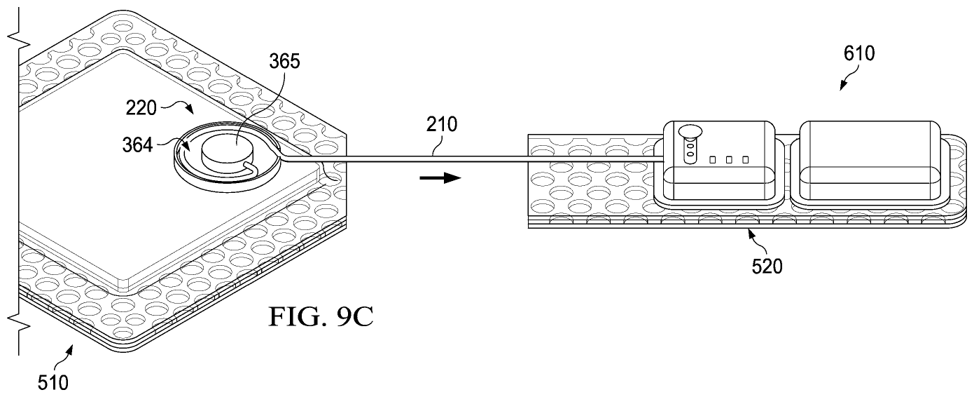

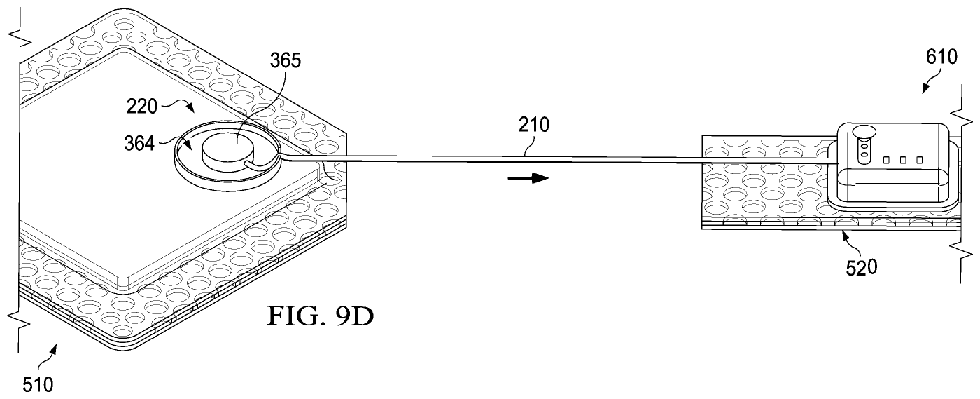

Additionally, the conduit 210 may be extended from the conduit chamber 220 to a length sufficient to provide fluid connection to the negative-pressure unit 610, particularly, the pump 730, and the dressing 110, particularly, the enclosure 342. For example, referring to FIGS. 9A, 9B, 9C, and 9D, the dressing 110 and negative-pressure unit 610 are shown with the conduit 210 extended from the conduit chamber 220 at varying lengths to enable fluid communication between the dressing 110 and the negative-pressure unit 610 when the negative-pressure unit 610 is positioned at various distances from the dressing 110. In FIGS. 9A, 9B, 9C, and 9D the conduit chamber cover 360 is shown removed in order to illustrate the disposition of the conduit 210 with respect to the conduit chamber 220. Where the conduit 210 is extended from the conduit chamber 220, a space between the conduit chamber cover 360 and the conduit interface 365 may allow the conduit 210 to be unspooled from around the conduit interface 365 and, thus, extended from the conduit chamber 220.

For example, in FIG. 9A the dressing portion 510 and tail portion 520 are shown attached and the negative-pressure unit 610 is shown in relatively-close proximity to the dressing portion 510. Also in FIG. 9A, the portion of the conduit 210 retained within the conduit chamber 220 remains within the conduit chamber 220, for example, such that a relatively small proportion of the conduit 210 extends from the conduit chamber 220.

Also for example, in FIGS. 9B and 9C the dressing portion 510 and tail portion 520 are shown separated and the negative-pressure unit 610 is shown positioned at relatively-intermediate proximities to the dressing portion 510. Also in FIGS. 9B and 9C, some of the portion of the conduit 210 originally retained within the conduit chamber 220 has been extended from the conduit chamber 220 and some of the portion of the conduit 210 originally retained within the conduit chamber 220 remains within the conduit chamber 220, for example, such that a relatively intermediate proportion of the conduit 210 extends from the conduit chamber 220.

Also for example, in FIG. 9D the dressing portion 510 and tail portion 520 are shown separated and the negative-pressure unit 610 is shown positioned at relatively-distant proximity to the dressing portion 510. Also in FIG. 9D, substantially all of the portion of the conduit 210 originally retained within the conduit chamber 220 has been extended from the conduit chamber 220, for example, such that a substantially all of the conduit 210 extends from the conduit chamber 220.

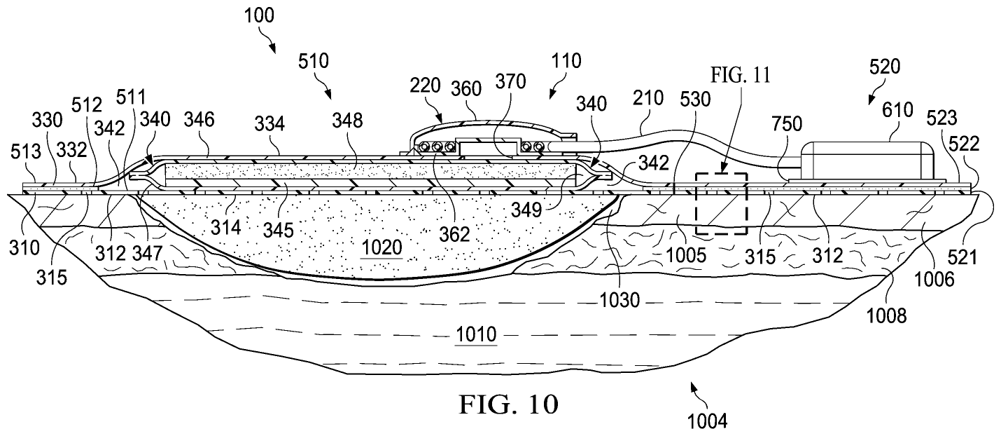

A system comprising the dressing 110 and negative-pressure unit 610 may be advantageously employed to provide negative-pressure therapy to a user. For example, FIG. 10 depicts an embodiment of the system 100 implemented in the treatment of a tissue site 1004 of a patient. The tissue site 1004 may extend through or otherwise involve an epidermis 1006, a dermis 1008, and a subcutaneous tissue 1010. In some embodiments, the tissue site 1004 may include a sub-surface portion that extends below the surface of the epidermis 1006. Additionally or alternatively, in some embodiments, the tissue site 1004 may include a surface portion that predominantly resides on the surface of the epidermis 1006, such as, for example, an incision. The system 100 may provide therapy to, for example, the epidermis 1006, the dermis 1008, and the subcutaneous tissue 1010, regardless of the positioning of the system 100 or the type of tissue site. The system 100 may also be utilized without limitation at other tissue sites.

In some embodiments, for example, the embodiment of FIG. 10, the system 100 may include an optional tissue interface component, such as an interface manifold 1020. The interface manifold 1020 is an optional component that may be omitted for different types of tissue sites or different types of therapy using reduced pressure, such as, for example, epithelialization, tissue closure, incision treatment, and others. If present, the interface manifold 1020 may be adapted to be positioned proximate to or adjacent to the tissue site 1004, such as, for example, by cutting or otherwise shaping the interface manifold 1020 in any suitable manner to fit the tissue site 1004. The interface manifold 1020 may be adapted to be positioned in fluid communication with the tissue site 1004 to distribute reduced pressure to the tissue site 1004. In some embodiments, the interface manifold 1020 may be positioned in direct contact with the tissue site 1004. The tissue interface or the interface manifold 1020 may be formed from any manifold material or flexible bolster material that provides a vacuum space, or treatment space, such as, for example, a porous and permeable foam or foam-like material, a member formed with pathways, a graft, or a gauze. As a more specific, non-limiting example, the interface manifold 1020 may be a reticulated, open-cell polyurethane or polyether foam that allows good permeability of fluids while under a reduced pressure. One such foam material is used in the V.A.C.® GRANUFOAM™ Dressing available from Kinetic Concepts, Inc. (KCI) of San Antonio, Texas. A material with a higher or lower density than the material of the V.A.C.® GRANUFOAM™ Dressing may be desirable for the interface manifold 1020 depending on the application. Among the many possible materials, the following may be used: the material in the V.A.C.® GRANUFOAM™ Dressing, a molded bed of nails structures, a patterned grid material such as those manufactured by Sercol Industrial Fabrics, 3D textiles such as those manufactured by Baltex of Derby, U.K., a gauze, a flexible channel-containing member, a graft, etc. In some instances, ionic silver may be added to the interface manifold 1020 by, for example, a micro bonding process. Other substances, such as anti-microbial agents, may be added to the interface manifold 1020 as well. In some embodiments, the interface manifold 1020 may comprise a porous, hydrophobic material. The hydrophobic characteristics of the interface manifold 1020 may prevent the interface manifold 1020 from directly absorbing fluid, such as exudate, from the tissue site 1004, but allow the fluid to pass through.

The dressing 110 may be positioned with respect to the tissue site 1004 such that the central portion 314 of the contact layer 310 is positioned at or proximate to the tissue site 1004, and such that the periphery 312 of the contact layer 310 is positioned proximate to peripheral tissue 1005 surrounding the tissue site 1004. In this manner, for example, the periphery 312 of the contact layer 310 may surround the interface manifold 1020. Further, the apertures 315 in the contact layer 310 may be in fluid communication with the interface manifold 1020 and tissue surrounding the tissue site 1004.

The cover 125 may cover the interface manifold 1020, the contact layer 310, the tissue site 1004, and the optional interface manifold 1020 to provide a fluid seal and a sealed space 1030 between the tissue site 1004 and the cover 125 of the dressing 110. Further, the cover 125 may cover other tissue, such as a portion of the epidermis 1006, surrounding the tissue site 1004 to provide the fluid seal between the cover 125 and the tissue site 1004. In some embodiments, a portion of the periphery 332 of the cover 125 may extend beyond the periphery 312 of the contact layer 310 and into direct contact with tissue surrounding the tissue site 1004. In other embodiments, the periphery 332 of the cover 125, for example, may be positioned in contact with tissue surrounding the tissue site 1004 to provide the sealed space 1030 without the contact layer 310. Thus, the adhesive 320 may also be positioned at least between the periphery 332 of the cover 125 and tissue, such as the epidermis 1006, surrounding the tissue site 1004. The adhesive 320 may be disposed on a surface of the cover 125 adapted to face the tissue site 1004 and the contact layer 310.

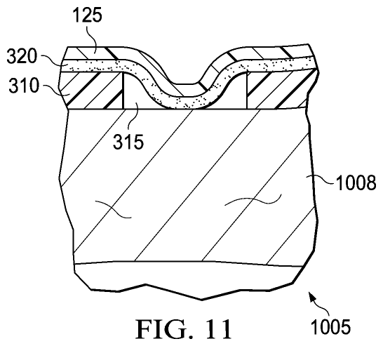

FIG. 11 is a detail view of a portion of FIG. 10. As shown in the embodiment of FIG. 11, the adhesive 320 may extend through or be pressed through the one or more of the plurality of apertures 315, for example so as to contact the epidermis 1006 and secure the dressing 110 to tissue at or surrounding the tissue site 1004 when the dressing 110 is positioned with respect to the tissue site 1004. For example, the apertures 315 may provide sufficient contact of the adhesive 320 to the epidermis 1006 to secure the dressing 110 with respect to the tissue site 1004. Additionally, the configuration of the apertures 315 and the adhesive 320 may also permit release and repositioning of the dressing 110 with respect to the tissue site 1004. In various embodiments, one or more of the apertures 315 may be adjusted in size and number to adjust the surface area of the adhesive 320 in fluid communication through the apertures 315, for example, for a particular application or geometry of the contact layer 310.

Additionally, in some embodiments, the adhesive 320 may be in fluid communication with the edges 319 through the apertures 315 being exposed at the edges 319. In this manner, the apertures 315 at the edges 319 may permit the adhesive 320 to flow around the edges 319 for enhancing the adhesion of the edges 319 around the tissue site, for example. The adhesive 320 may be in fluid communication with the apertures 315 in at least the periphery 312 of the contact layer 310. In this manner, the adhesive 320 may be in fluid communication with the tissue surrounding the tissue site through the apertures 315 in the contact layer 310.

In use, the desired placement of the dressing 110 and the negative-pressure unit 610 may be determined to meet the specific needs of a therapy. Where it is desirable for the negative-pressure unit 610 to be placed adjacent to the tissue site 1004 that will receive therapy, the dressing 110 may be placed with respect to the tissue site 1004 with the dressing intact, for example, without separating the dressing portion 510 and the tail portion 520. The dressing 110 may be placed with respect to the tissue site 1004 with the dressing portion 510 and the tail portion 520 intact.

Alternatively, wherein it is desirable for the negative-pressure unit 610 to be placed further from the tissue site 1004 that will receive therapy, the dressing 110 may be placed with respect to the tissue site 1004 with the dressing with the dressing portion 510 separated from the tail portion 520, for example, by cutting or tearing via the cut-line 530. The dressing portion 510 may be placed with respect to the tissue site 1004 and the tail portion 520 may be attached at a location proximate to the dressing portion 510, for example, not more than about 400 mm from the dressing portion 510, or, not more than about 350 mm from the dressing portion 510, or, not more than about 300 mm from the dressing portion 510. With the dressing portion 510 and tail portion 520 placed, the negative-pressure unit 610 may be affixed to the tail portion 520, for example, by fixing a layer of the reclosable fastener 750 fixed to the negative-pressure unit 610 to another layer of the reclosable fastener 750 fixed to the tail portion 520.

With the negative-pressure unit 610 affixed to the tail portion 520, and thus secured to the user, the conduit 210 may provide a route of fluid communication between the negative-pressure unit 610 and the dressing 110. In some embodiments, for example, dependent upon the location of the negative-pressure unit 610 with respect to the dressing portion 510, it may be necessary to extend some portion of the conduit 210 from the conduit chamber 220 to enable connection to the negative-pressure unit 610, for example, by pulling a length of the conduit 210 from the conduit chamber 220.

With the negative-pressure unit 610 secured to the user and placed in fluid communication with the dressing, the negative-pressure unit 610 may be operated to provide negative-pressure therapy to the tissue site 1004. During the course of the negative-pressure therapy, it may become desirable or necessary to replace the dressing 110. In such embodiments, the negative-pressure unit 610 may be disconnected from the conduit 210 associated with the original dressing 110, the original dressing 110 removed, and a replacement dressing 110 likewise placed with respect to the tissue site 1004. The negative-pressure unit 610 may be similarly affixed to the tail portion 520 of the replacement dressing 110 and fluidly coupled with the conduit 210 associated with the replacement dressing 110. Additionally, it may be necessary to replace or recharge the battery 720, for example, dependent upon the duration over which the negative-pressure unit 610 has been previously used. As such, the negative-pressure unit 610 may advantageously be reused with multiple dressings 110.

Thus, in some embodiments, the dressing 110 may be wearable, for example, as a single-component “peel-and-place” dressing. The dressing 110 may be customized based upon the needs of a particular therapeutic environment, for example, particular to the location of a tissue site that will receive the therapy. For example, the dressing 110 may provide an integrated negative-pressure conduit capable of being extended to various lengths, enabling the dressing portion 510 to be placed with respect to the tissue site that will receive therapy while the tail portion 520 is affixed at a desired location, enabling the negative-pressure unit 610 to be worn by the user at a desired location. The dressing may also allow the negative-pressure unit 610 to be affixed to the user via a “skin-friendly,” low-trauma interface, which may lessen the possibility of any trauma associated with removable the system or components thereof. Additionally, the system allows for the negative-pressure unit 610, to be removed and reused in multiple iterations.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An apparatus for treating a tissue site with negative pressure, the apparatus comprising:
a dressing;
a dressing interface fluidly coupled to the dressing;
a conduit comprising a first end and a second end, wherein the first end is fluidly coupled to the dressing interface; and
a conduit chamber configured to retain at least a portion of the conduit and to allow the second end of the conduit to be extended from the dressing interface.

2. The apparatus of claim 1, wherein the dressing comprises:
a contact layer having a dressing portion and a tail portion adjacent to the dressing portion;
a manifold disposed adjacent to the dressing portion; and
a dressing cover disposed over the manifold and adhered to the dressing portion and the tail portion.

3. (canceled)

4. (canceled)

5. The apparatus of claim 2,
wherein the contact layer comprises a plurality of apertures, and
wherein an adhesive is exposed through the plurality of apertures.

6. The apparatus of claim 5, wherein the contact layer comprises silicone gel and the adhesive is an acrylic adhesive.

7. The apparatus of claim 1, further comprising a conduit chamber cover disposed over the dressing interface to form the conduit chamber.

8. The apparatus of claim 7, wherein the portion of the conduit retained within the conduit chamber is disposed in the conduit chamber in a coiled pattern.

9. The apparatus of claim 7, wherein the portion of the conduit retained within the conduit chamber is disposed in the conduit chamber in a serpentine pattern.

10. (canceled)

11. The apparatus of claim 2, further comprising a negative-pressure source configured to be fluidly coupled to the second end of the conduit and removably attached to the tail portion.

12. The apparatus of claim 1, further comprising:
a reclosable fastener adhered to the dressing;
a housing mounted to the reclosable fastener, the housing comprising a battery chamber, a pump chamber, and a port configured to receive the second end of the conduit;
a pump disposed in the pump chamber, the pump configured to be electrically coupled to a battery in the battery chamber and to deliver negative pressure to the port; and
at least one cap disposed over the pump chamber and the battery chamber.

13.-17. (canceled)

18. A therapy unit for treating a tissue site with negative pressure, the therapy unit comprising:
a medical drape with at least one adhesive, the medical drape being connected to a first portion of a reclosable fastener;
a housing mounted to a second portion of a reclosable fastener, the housing comprising; and
a pump configured to be electrically coupled to a battery and to a negative pressure port.

19.-21. (canceled)

22. The therapy unit of claim 18, wherein the at least one adhesive includes a first adhesive comprising a silicone adhesive and second adhesive comprising an acrylic adhesive.

23. (canceled)

24. The therapy unit of claim 18, further comprising a negative pressure conduit coupled to the housing.

25. The therapy unit of claim 24, wherein the medical drape is fluidly coupled to the pump.

26. The therapy unit of claim 18, wherein:
a first layer of the reflowable fastener comprises a first array of stems;
a second layer of the reclosable fastener comprises a second array of stems; and
the first array of stems is configured to interlock with the second array of stems.

27. The therapy unit of claim 18, wherein
the reclosable fastener comprises a layer of hooks and a layer of loops; and
the layer of hooks is configured to interlock with the layer of loops.

28. The therapy unit of claim 18, wherein the reclosable fastener comprises a pressure-sensitive adhesive.

29. (canceled)

30. The therapy unit of claim 18, wherein a first layer of the reclosable fastener, a second layer of the reclosable fastener, or both the first layer and the second layer includes an array of perforations.

31. A system for treating a tissue site with negative pressure, the system comprising:
a dressing;
a dressing interface fluidly coupled to the dressing;
a therapy unit; and
a conduit configured to couple the dressing interface to the therapy unit and to allow the therapy unit to be placed adjacent to the dressing within a range of about 40 millimeters to about 500 millimeters of the dressing interface.

32. (canceled)

33. The system of claim 31, wherein the dressing comprises:
a contact layer having a dressing portion and a tail portion adjacent to the dressing portion;
a manifold disposed adjacent to the dressing portion; and
a dressing cover disposed over the manifold and adhered to the dressing portion and the tail portion.

34. The system of claim 33, wherein:
the therapy unit is adhered to the dressing cover opposite to the tail portion,
the dressing further comprises an attachment device configured to adhere the contact layer to an attachment surface;
the therapy unit is adhered to the dressing cover with a first bond strength;
the attachment device is configured to adhere the contact layer to the attachment surface with a second bond strength; and
the first bond strength is less than the second bond strength.

35.-46. (canceled)
